(19) United States
(12) Patent Application Publication
(10) Pub. No.: US 2007/0055328 A1
(43) Pub. Date: Mar. 8, 2007

(54) DEVICE AND METHOD FOR ESOPHAGEAL COOLING

(76) Inventors: MARTIN L. MAYSE, University City, MO (US); James P. Hummel, East Haven, CT (US)

Correspondence Address: CHARLES C. MCCLOSKEY, 763 S. NEW BALLAS ROAD STE. 170, ST. LOUIS, MO, 63141, US

(21) Appl. No.: 11/469,749

(22) Filed: Sep. 1, 2006

Related U.S. Application Data
(60) Provisional application No. 60/713,301, filed on Sep. 2, 2005.

(51) Int. Cl. A61F 7/00; A61F 7/12

(52) U.S. Cl. 607/105; 607/113

(57) ABSTRACT

The present invention includes a device and a method for preventing injury of the esophagus during thermal ablation of the left atrium. The device has an esophageal probe with a balloon tip for insertion into the esophagus of a patient. During usage, coolant passes into the esophageal probe and then fills its balloon. The coolant, when circulating through the balloon and an external cooling machine, protects the esophageal tissue in contact with the esophageal probe from thermal damage during ablation of the posterior wall of the left atrium of the heart, or other procedure.

### CROSS REFERENCE TO RELATED APPLICATION

This non-provisional application claims priority to the provisional application for patent Ser. No. 60/713,301 which was filed on Sep. 2, 2005 which is incorporated by reference and the aforesaid application is commonly owned by the same inventors.

### BACKGROUND OF THE INVENTION

The present invention generally relates to medical devices utilized in protecting the digestive tract of a person. More specifically, the present invention relates to an esophageal probe with a cooled tip that protects the esophagus when the adjacent left atrium of the heart is ablated.

Cardiac arrhythmias generally require a critical anatomic region of abnormal impulse formation, or propagation, to initiate or sustain themselves. If the ablation can alter, or destroy, this critical region, the arrhythmia ceases. Potential energy sources for ablation include radiofrequency, ultrasound, microwave, laser, cryothermy, and other electromagnetic radiation. These modalities may be applied endocardially or epicardially by either a percutaneous or surgical approach.

One risk of thermal injury to the myocardium by any ablation is collateral damage to nearby structures in the body of a patient. Potential complications associated with thermal ablation of heart tissue include injury to the coronary arteries, phrenic nerve, lung, aorta, esophagus, or other thoracic structures.

Radiofrequency is currently the most common source of energy for catheter ablation of cardiac arrhythmias. The flow of radiofrequency current through myocardial tissue causes resistive heating at the electrode-tissue interface. Direct resistive heating depends on the power density within the tissue, which decreases in proportion to the distance from the ablation electrode. Thus the depth of tissue which is heated resistively is generally less than 2 mm. Thermal injury to deeper myocardium, as well as any contiguous noncardiac structure, occurs by heat conduction.

Atrial fibrillation is the most common sustained arrhythmia present in humans, occurring in 0.4-0.9% of the general population and 3-4% of those over the age of 60. Atrial fibrillation has significant patient morbidity and mortality, as well as economic cost. In recent years, radiofrequency ablation has become an important alternative to anti-arrhythmic therapy for atrial fibrillation. The pulmonary veins and the posterior left atrium are critical areas in the initiation and maintenance of atrial fibrillation in many patients. Radiofrequency ablation around the pulmonary veins and in the posterior atrium has effectively treated atrial fibrillation (Oral H., et al. Circulation 2003).

A potential complication of performing ablation in this region of the left atrium, however, is causing damage to the esophagus which is in close proximity to the posterior wall. Conduction of heat to the esophagus from a nearby endocardial lesion site has caused several fatal atrio-esophageal fistulas following atrial fibrillation ablation (Pappone C., et al. Circulation 2004).

Several strategies have been employed to avoid this potentially catastrophic complication. Some physicians have reduced the amount of power delivered to this area, or tried to avoid ablation in the posterior atrium altogether. However, the posterior left atrium appears to be a critical region in the initiation and maintenance of atrial fibrillation in many patients, and thus is likely a necessary target of any efficacious ablation approach. Other physicians have begun using esophageal temperature monitoring during ablation. If a rise in temperature is detected in the esophagus, the ablation lesion is terminated. However, simply monitoring temperature at some position within the lumen of the esophagus may not reliably prevent injury. If the endocardial ablation site and contiguous esophageal tissue are at some distance from the temperature sensor, the extent of thermal injury may not be appreciated.

The present invention seeks to prevent thermal injury to the esophagus during ablation by cooling the esophageal tissue just prior to and during ablation

### SUMMARY OF THE INVENTION

The cooling device of the present invention is an esophageal probe with a balloon tip for insertion into the esophagus of a patient. Coolant enters the esophageal probe and fills the balloon tip of the esophageal probe. During ablation, any heat conducted from the heart into the contiguous structure, particularly esophageal tissue, would rapidly dissipate by the coolant in the balloon. Thus, the coolant protects the esophageal tissue in contact with the probe from thermal damage during ablation of the posterior wall of the left atrium of the heart.

It is, therefore, the principal object of this invention to provide thermal protection to tissues in the proximity of an ablation.

Another object of this invention is to provide a stable temperature environment while an ablation is performed.

A further object of this invention is to provide a complete connection around the perimeter of the invention to the surrounding tissue resulting in even distribution of temperature protection.

A further object of this invention is to provide a pressurized coolant within safe limits for the tissue proximate to an ablation.

These and other objects may become more apparent to those skilled in the art upon review of the summary of the invention as provided herein. In addition, the invention will be better understood upon undertaking a study of the description of its preferred embodiment, in view of the drawings.

### BRIEF DESCRIPTION OF THE DRAWINGS

In referring to the drawings,

FIG. 1A is a partial front sectional view of the human body illustrating the position of the heart;

FIG. 1B is a cross sectional view of the human body, at the seventh thoracic vertebra illustrating the relative position of the left atrium of the heart and the esophagus;

FIG. 2 is a partial sectional view of the human body taken along the mid-sagittal plane;

FIG. 3A is a longitudinal side view of the esophageal probe of the present invention;

FIG. 3B is a cross sectional view of the esophageal probe of the present invention;

FIG. 3C is a cross sectional view of the esophageal probe of the present invention;

FIG. 3D is a partial longitudinal sectional view of the esophageal probe taken through the balloon and the distal portions of the coolant in-flow and out-flow lines; and,

FIG. 4 is a partial sectional view of the human body taken along the mid-sagittal plane showing the relative position of the left atrium of the heart and esophagus with the esophageal probe in place and the balloon inflated and in contact with the esophagus near the left atrium of the heart.

The same reference numerals refer to the same parts throughout the various figures.

### DESCRIPTION OF THE PREFERRED EMBODIMENT

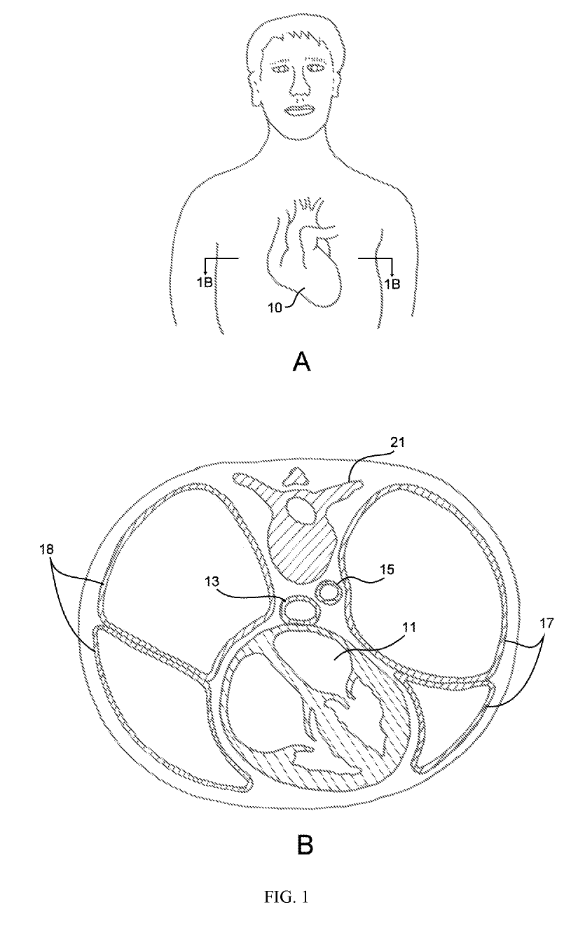

In reference to the drawings, FIG. 1A is a partial front sectional view of the human body illustrating the position of the heart 10 within the chest. FIG. 1B is a cross sectional view of the human body, at the level of the seventh thoracic vertebra 21 illustrating the relative position of the left atrium 11 of the heart 10 and the esophagus 13. It should be noted from FIGS. 1A and 1B, that the esophagus 13 is essentially in direct contact with the left atrium 11 for a portion of its course through the chest. The esophagus 13 is also flanked by the left lung 17 and right lung 18. The aorta 15 is positioned between the esophagus 13 and the left lung 17 and is in close proximity to the thoracic vertebra 21. It is well demonstrated in FIG. 1B that application of thermal energy to the posterior wall of the left atrium 11 of the heart 10 can potentially injure the anterior wall of the adjacent esophagus 13.

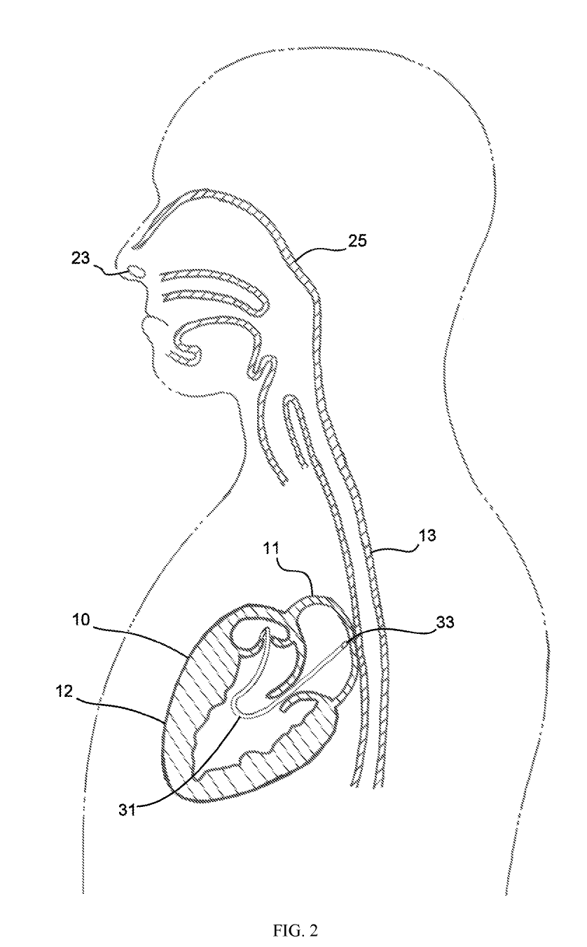

FIG. 2 is a partial longitudinal sectional view of the human body taken along the mid-sagittal plane and again demonstrates the relative position of the left atrium 11 of the heart 10 and esophagus 13, the nasal passage 23 and the pharynx 25. An ablation catheter 31 is shown passing through the left ventricle 12 and into the left atrium 11 of the heart 10. The tip 33 of the ablation catheter 31 is shown in contact with the posterior wall of the left atrium 11 of the heart 13.

During a prior art therapy session utilizing thermal ablation to treat atrial fibrillation, a therapist would direct the ablation catheter 31 such that thermal energy would pass from the catheter tip 33 and into the tissues of the posterior wall of the left atrium 11. Heating of the posterior wall of the left atrium 11 would then occur, ideally leading to localized injury of the left atrium 11 and resolution of atrial fibrillation. Depending upon the type of ablation catheter used, the length of the therapy session, and the amount of energy supplied to catheter tip 33, tissue heating could extend beyond the posterior wall of the left atrium 11 and encompass the anterior wall of the esophagus 13. As is well known, due to the close proximity of the esophagus 13 to the left atrium 11, the esophagus 13 can be injured during thermal ablation of the posterior wall of the left atrium 11. Should this occur, it is possible for a fistula tract to form between the left atrium 11 and the esophagus 13, and death can ensue from massive bleeding. This possible complication has led to many therapists avoiding the posterior wall of the left atrium 11 during therapy to minimize the risk of injury to the esophagus 13. This prior art approach also tends to decrease the effectiveness of ablation in the treatment of atrial fibrillation. The present invention is a balloon tipped esophageal probe that provides a means to cool the esophagus 13 during thermal ablation of the left atrium 11 and thus minimize the possibility of developing a fistula tract between the left atrium 11 and the esophagus 13.

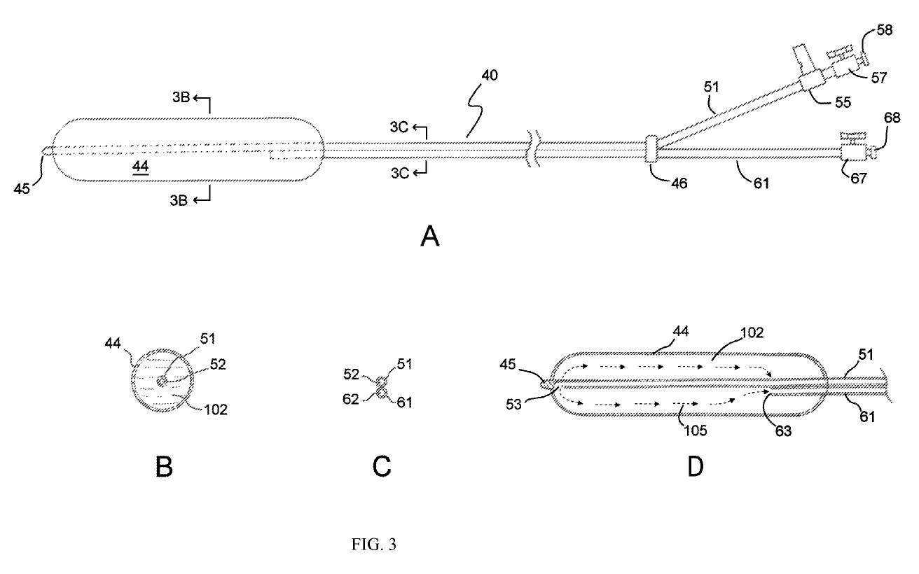

FIG. 3A demonstrates a longitudinal side view of the esophageal probe 40 of the present invention. FIG. 3B demonstrates a cross sectional view of the esophageal probe 40 of the present invention. This view shows the balloon 44 with the coolant contained therein and the inflow line 51 admitting additional coolant through its lumen 52. Then, FIG. 3C demonstrates a cross sectional view of the esophageal probe 40 of the present invention. This view shows the in-flow line 51 as contiguous with the out flow line 61 from the junction, as at 46, to the interior of the balloon 44. And, FIG. 3D demonstrates a partial longitudinal sectional view of the esophageal probe 40 taken through the balloon 44 with lines of flow 105 demonstrating the movement of coolant through the coolant volume 102 within the expanded balloon 44.

The principal components of the esophageal probe 40 include a distensible, thermally conductive balloon 44, a coolant in-flow line 51, and a coolant out-flow line 61. The proximal end of the coolant in-flow line 51 has an inline in-flow valve 57 and pressure relief valve 55. The proximal end of the coolant out-flow line 61 also has an out-flow valve 67. During use, the coolant in-flow line 51 can be connected to a coolant source by means of the connector 58. The lumen 52 of the coolant in-flow line 51 and the lumen 62 of the coolant out-flow line 61 provide a path for coolant to be transmitted from the coolant supply to coolant space 102 of the balloon 44 and then out through another connector 68 for additional cooling by the coolant supply.

The balloon 44 may be composed of any distensible, chemically inert, non-toxic and thermally conductive material. The coolant in-flow line 51 and the coolant out-flow line 61 may be composed of any suitable flexible, chemically inert, non-toxic material for withstanding operating pressures without significant expansion. The coolant in-flow line 51 and the coolant out-flow line 61 have suitable length for placement in the esophagus 13 near the left atrium 11 of the heart 10, approximately 80 cm. The coolant in-flow line 51 and the coolant out-flow line 61 may desirably have markings or other indicator (not shown) along their length to indicate distance there-along so that the balloon 44 may be initially positioned approximately adjacent the left atrium 11.

Though inflow line 51 and outflow line 61 are contiguous, FIG. 3D shows the measures taken to prevent cross connection, siphoning, or back flow between the two lines within the balloon 44. The inflow line 51 enters one end of the balloon 44 and extends through the length of the balloon. The inflow line 51 reaches the opposite end of the balloon and connects to the balloon. Opposite the ending of the inflow line, the balloon has the tip 45. With the tip opposite the inflow line, the tip transmits maximum cooling by conduction when the tip is placed upon a point within the body. Near the tip 45, the inflow line 51 has an aperture 53 that releases coolant into the balloon 44. The coolant flows within the balloon and then is collected into the outflow line 61 at its opening 63. The opening 63 is generally at the end of the outflow line 61 and collects coolant from any direction.

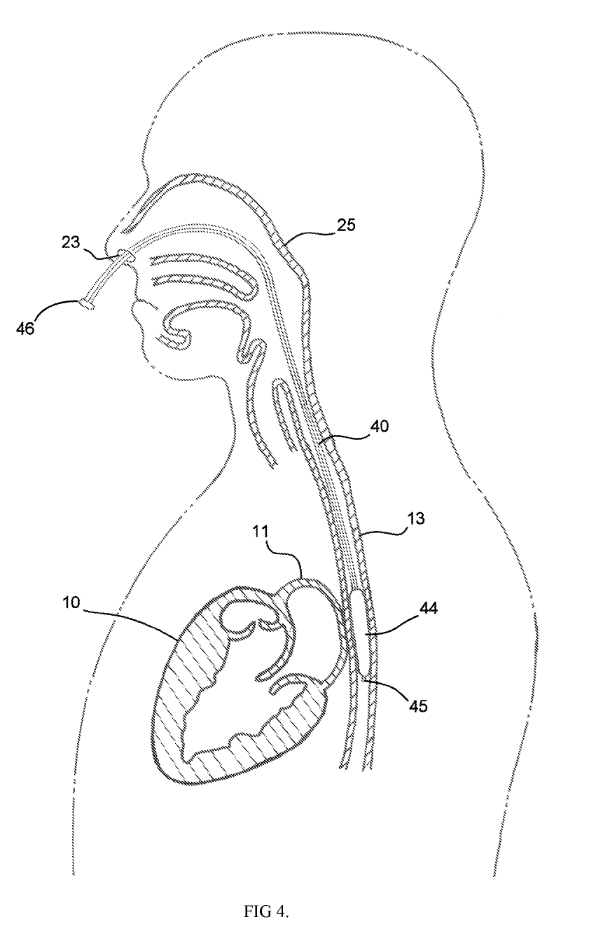

FIG. 4 is a partial sectional view of the human body taken along the mid-sagittal plane showing the relative position of the left atrium 11 of the heart 10 and esophagus 13 with the esophageal probe 40 in place and the balloon 44 inflated and in contact with the esophagus 13 near the left atrium 11 of the heart 10.

Referring to FIG. 4, an esophageal probe 40 with the balloon 44 fits within the esophagus 13 of a human body for the purpose of protecting the anterior wall of the esophagus 13 from thermal injury that may occur during thermal ablation of the left atrium 11 of the heart 10. The esophageal probe 40 is inserted tip 45 first through the nasal passage 23, through the pharynx 25, and then into the esophagus 13. Alternatively, the esophageal probe 40 may be inserted through the mouth of the patient.

Once the esophageal probe 40 is properly inserted into the esophagus 13, but prior to energizing the ablation catheter 31, (shown earlier in FIG. 2), the balloon 44 is filled with coolant from the in-flow line 51 until the balloon 44 properly occupies width of the esophagus 13, but does not overly distend the esophagus 13 proximate the left atrium 11. The coolant that fills and circulates through the balloon 44 maintains the temperature of the anterior wall of the esophagus 13 within physiologically normal temperature ranges and thus prevents esophageal injury. Proper inflation establishes substantially complete contact with the esophageal wall to prevent “hot spots” from occurring adjacent to the esophageal wall during thermal ablation while also ensuring that the esophagus 13 is not ruptured due to improper pressure against the esophageal wall.

The balloon 44 of the esophageal probe 40 fills with coolant in a various ways. In one method, the coolant out-flow valve 67 is closed and the coolant in-flow valve 57 is opened. Coolant is able to pass through the coolant in-flow valve 57, through the pressure relief valve 55, down the lumen 52 of the coolant in-flow line 51 and into the balloon 44. Coolant is prevented from leaving the balloon 44 by the closed coolant out-flow valve 67. Coolant flow continues until the pressure within the balloon 44 equals the pressure in the coolant source or the coolant in-flow valve 57 is closed. The pressure relief valve 55 limits the maximum pressure in the coolant in-flow line 51, by releasing coolant from the balloon 44 should the pressure in the coolant in-flow line 51 rise above a certain predetermined safe level pertinent to the surrounding tissue.

With both the coolant in-flow valve 57 and the coolant out-flow valve 67 open, coolant is able to flow continuously from the coolant source through the coolant in-flow valve 57, through the pressure relief valve 55, down the lumen 52 of the coolant in-flow line 51, into and through the balloon 44 and out through the lumen 62 of the coolant out-flow line 61 and the coolant out-flow line valve 67. The flow lines 105 in FIG. 3D represent coolant flow through the coolant area 102 of the balloon 44. The rate of coolant flow into the probe is controlled by adjusting the pressure within the coolant source and the positions of the coolant in-flow valve 57 and the coolant out-flow valve 67.

The esophageal probe 40 of the present invention is intended for use with any of a variety of thermal ablation catheters 31. The esophageal probe 40 provides coolant to the balloon 44 located in the esophagus 13 near the left atrium 11. This coolant will prevent the temperature of the anterior esophageal 13 wall from increasing above a predetermined temperature during thermal ablation of the left atrium 11. By supplying coolant to the balloon 44, the esophageal probe 40 also modifies the heating pattern caused by the thermal ablation catheter 31. In particular, the heating pattern no longer encompasses the esophagus 13 and a greater portion of the tissue in the posterior wall of the left atrium 11 can be ablated while adjacent healthy esophageal 13 wall is protected.

The present invention also provides a means for pre-chilling the anterior esophageal wall prior to applying energy from the catheter tip 33 to the left atrium 11. The use of pre-chilling would permit the therapist to more quickly increase the energy to the thermal ablation catheter 31 without damaging esophageal 13 tissue.

Variations or modifications of the subject matter of this invention may occur to those skilled in the art upon reviewing the disclosure provided herein. Such variations or modifications are intended to be encompassed within the scope of the invention as described herein. The description of the preferred embodiment and of the drawings showing the same are provided herein for illustrative purposes only.

From the aforementioned description, a device and method for esophageal cooling has been described. The esophageal cooling device is uniquely capable of readily protecting the esophagus when the adjacent left atrium is ablated. The cooling device and its various components may be manufactured from many materials including but not limited to polymers, silicone, high density polyethylene HDPE, polypropylene PP, polyethylene terephalate ethylene PETE, polyvinyl chloride PVC, nylon, ferrous and non-ferrous metals, their alloys and composites.

The phraseology and terminology employed herein are for the purpose of description and should not be regarded as limiting. As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

I claim:

1. A device for cooling a passage and adjacent tissue within the body of a person using externally supplied coolant, comprising:
a flexible and collapsible container;
an inflow line entering said container and admitting coolant therein;
an outflow line exiting said container, releasing coolant therefrom, and generally contiguous with said inflow line; and,
said container locating within a passage adjacent to a portion of the body undergoing medical treatment at a higher temperature.

2. The cooling device of claim 1 further comprising:
said container being an elongated balloon with two opposite ends.

3. The cooling device of claim 2 wherein said balloon has a tip upon one end.

4. The cooling device of claim 1 further comprising:
said inflow line extending the length of said container, approaching the opposite end of said container from where said inflow line enters said container, and having an aperture for releasing coolant proximate the opposite end; and,
said inflow line extending away from said container a sufficient length for inserting said device into the body of a person.

5. The cooling device of claim 4 wherein said aperture is upon the side of said inflow line.

6. The cooling device of claim 4 further comprising:
said inflow line having an inflow valve opposite said container.

7. The cooling device of claim 5 further comprising:
said inflow line having a pressure relief valve away from said inflow valve and towards said container, said pressure relief valve having an upper limit suitable for said passage and said tissue, and a connector outside said inflow valve for supplying coolant to said inflow line.

8. The cooling device of claim 1 further comprising:
said outflow line extending partially into said container, and having an aperture for collecting coolant, said aperture being perpendicular to the flow of coolant; and,
said outflow line extending away from said container a sufficient length for inserting said device into the body of a person.

9. The cooling device of claim 8 further comprising:
said outflow line having an outflow valve opposite said container.

10. The cooling device of claim 5 further comprising:
said outflow line having a connector outside said outflow valve for releasing coolant to the external coolant supply.

11. A method for cooling a passage and adjacent tissue within the body of a person, comprising:
inserting a container within said passage;
supplying a coolant to said container;
distributing said coolant throughout said container;
allowing said coolant to cool the passage and adjacent tissue;
collecting said coolant when warmed and returning said coolant to the supply; and,
withdrawing said container from said passage.

12. The method for cooling a passage of claim 11 further comprising:
said supplying coolant through an inflow line contiguous with an outflow line for said collecting said coolant.

13. The method for cooling a passage of claim 12 further comprising:
said distributing said coolant occurring as said inflow line and said outflow lines having different lengths within said container.

14. A device for cooling the esophagus of a person during ablation of the heart comprising:
a balloon locating in said esophagus proximate the left atrium of said heart;
a coolant circulating through said balloon through an inflow line and an outflow line; said coolant in said balloon preventing the temperature of the anterior esophageal wall from increasing during ablation of the left atrium; said coolant in said balloon modifying the distribution of heat by ablation to avoid the esophagus; and,
said balloon being wider than said inflow line and said outflow line combined.
